(19) United States
(12) Patent Application Publication
(10) Pub. No.: US 2025/0352223 A1
(43) Pub. Date: Nov. 20, 2025

(54) POSITIONING AND CUTTING GUIDES AND ASSOCIATED METHODS FOR ORTHOPEDIC PROCEDURES

(71) Applicant: Paragon 28, Inc., Englewood, CO (US)

(72) Inventors: Stormy HEGG, Denver, CO (US); Elliott ISCHE, Denver, CO (US); Steven SAID, Denver, CO (US); Michael SCHMIDT, Greenwood Village, CO (US); Laura Zagrocki BRINKER, Lone Tree, CO (US); Kaitlin KARAS, Denver, CO (US)

(73) Assignee: Paragon 28, Inc., Englewood, CO (US)

(21) Appl. No.: 19/211,925

(22) Filed: May 19, 2025

Related U.S. Application Data
(60) Provisional application No. 63/648,988, filed on May 17, 2024.

(51) Int. Cl. A61B 17/17; A61B 17/00

(52) U.S. Cl. CPC A61B 17/1775; A61B 17/1796; A61B 2017/00477

(57) ABSTRACT

An implant having a first end opposite a shaft from a second end, where the first end, the second end, and the shaft are all integral. The first end includes an opening in an end portion that extends along a longitudinal axis and is in fluid communication with the cannulations of the shaft and the second end. The second end includes a dynamic feature that is actuatable between two positions. The implant also includes an actuator extending from the first opening, along the cannulation of the shaft and into the cannulation of the second end. Manipulation of the actuator can bias the dynamic feature from the first position to the second position.

### CROSS RELATED APPLICATIONS

This application claims benefit of priority of U.S. Provisional Application No. 63/648,988 filed on May 17, 2024, and entitled “Positioning and Cutting Guides and Associated Methods For Orthopedic Procedures,” the disclosure of which is hereby incorporated herein by reference in its entirety.

The present disclosure relates to systems, instruments, and surgical methods to be implemented in surgical procedures. The present disclosure relates to podiatric and instruments and surgical methods to be implemented in various procedures of the foot, ankle, or other anatomy. More specifically, but not exclusively, the present disclosure relates to a system and associated methodology to be implemented procedures of the foot and/or ankle.

### BACKGROUND OF THE INVENTION

Many currently available surgical systems, instruments, and methods do not completely address the needs of patients. Additionally, many currently available surgical systems, instruments, and methods fail to account for properties of foot and ankle anatomy and accordingly can decrease favorability of the outcome for the patient.

### SUMMARY OF THE INVENTION

The present disclosure is directed toward systems, instruments, and surgical methods to be implemented in procedures of the foot and ankle.

One aspect of the present disclosure is directed to an orthopedic instrument system. The instrument system includes a first cutting guide, which has a coupling portion including a plurality of openings extending from a front surface therethrough to a rear surface, and a cutting portion integral with the coupling portion. The cutting portion includes a cannulation extending from a top surface therethrough to a bottom surface, and a window extending from the front surface through to the rear surface and overlapping with the cannulation. The system also includes a second cutting guide, which has a coupling portion including a plurality of openings extending from a front surface therethrough to a rear surface, and a cutting portion integral with the coupling portion. The cutting portion includes a cannulation extending from a top surface therethrough to a bottom surface, and a window extending from the front surface through to the rear surface and overlapping with the cannulation. The system also includes at least one insert receivable at least partially within the cannulation of the first and second guides, the insert including a slot extending therethrough.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is an elevated, front, perspective view of an exemplary orthopedic system, in accordance with the present disclosure;

FIG. 2 is an elevated, rear, perspective view of the exemplary orthopedic system of FIG. 1, in accordance with the present disclosure;

FIG. 3 is a front view of the exemplary orthopedic system of FIG. 1, in accordance with the present disclosure;

FIG. 4 is a front view of the exemplary orthopedic system of FIG. 1, in accordance with the present disclosure;

FIG. 5 is an alternate front perspective view of the exemplary orthopedic system of FIG. 1, in accordance with the present disclosure;

FIG. 6 is a front view of a portion of the exemplary orthopedic system of FIG. 1, in accordance with the present disclosure;

FIG. 7 is a front view of a component of the portion of the exemplary orthopedic system of FIG. 6, in accordance with the present disclosure;

FIG. 8 is a front perspective view of the portion of the exemplary orthopedic system of FIG. 6, in accordance with the present disclosure;

FIG. 9 is a rear perspective view of the portion of the exemplary orthopedic system of FIG. 6, in accordance with the present disclosure;

FIG. 10 is a top view of the portion of the exemplary orthopedic system of FIG. 6, in accordance with the present disclosure;

FIG. 11 is a bottom view of the portion of the exemplary orthopedic system of FIG. 6, in accordance with the present disclosure;

FIG. 12 is a front view of the portion of the exemplary orthopedic system of FIG. 6, in accordance with the present disclosure;

FIG. 13 is a rear view of the portion of the exemplary orthopedic system of FIG. 6, in accordance with the present disclosure;

FIG. 14 is a side view of the portion of the exemplary orthopedic system of FIG. 6, in accordance with the present disclosure;

FIG. 15 is an alternate side view of the portion of the exemplary orthopedic system of FIG. 6, in accordance with the present disclosure;

FIG. 16 is a front view of a portion of the exemplary orthopedic system of FIG. 1, in accordance with the present disclosure;

FIG. 17 is a front perspective view of the portion of the exemplary orthopedic system of FIG. 16, in accordance with the present disclosure;

FIG. 18 is a rear perspective view of the portion of the exemplary orthopedic system of FIG. 16, in accordance with the present disclosure;

FIG. 19 is a top view of the portion of the exemplary orthopedic system of FIG. 16, in accordance with the present disclosure;

FIG. 20 is a bottom view of the portion of the exemplary orthopedic system of FIG. 16, in accordance with the present disclosure;

FIG. 21 is a front view of the portion of the exemplary orthopedic system of FIG. 16, in accordance with the present disclosure;

FIG. 22 is a rear view of the portion of the exemplary orthopedic system of FIG. 16, in accordance with the present disclosure;

FIG. 23 is a side view of the portion of the exemplary orthopedic system of FIG. 16, in accordance with the present disclosure;

FIG. 24 is an alternate side view of the portion of the exemplary orthopedic

system of FIG. 16, in accordance with the present disclosure;

FIG. 25 is a front view of a component of the portion of the exemplary orthopedic system of FIG. 16, in accordance with the present disclosure; and

FIG. 26 is a front perspective view of a component which may be implemented with one or both of the portions of FIGS. 6 and/or 16 of the exemplary orthopedic system of FIG. 1, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

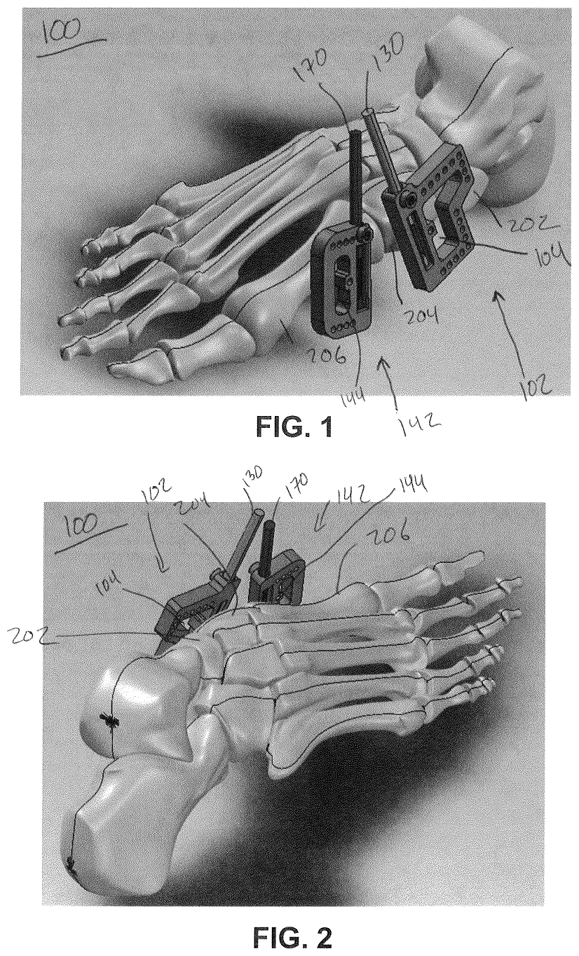

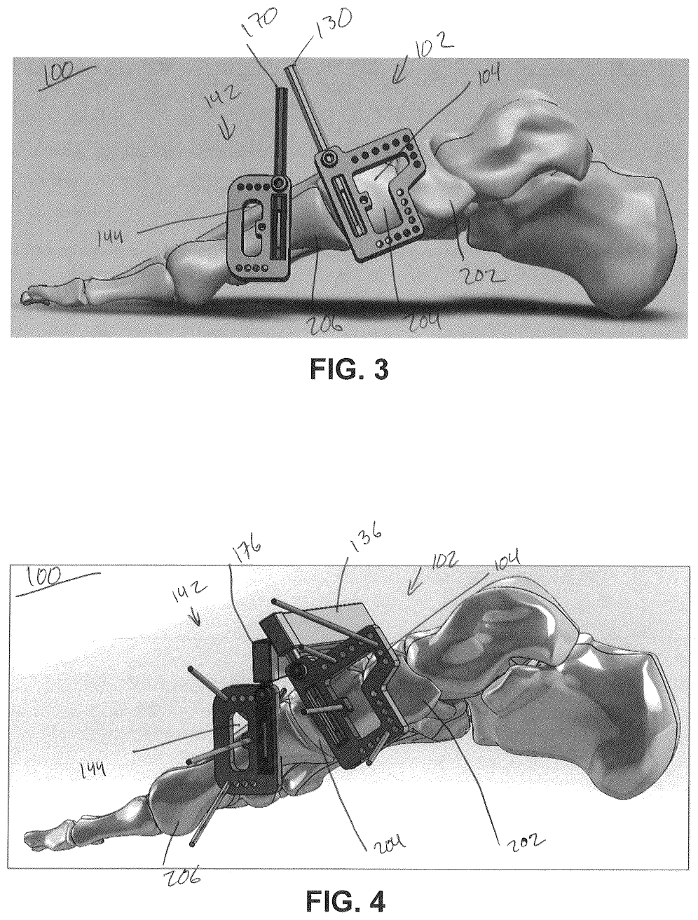

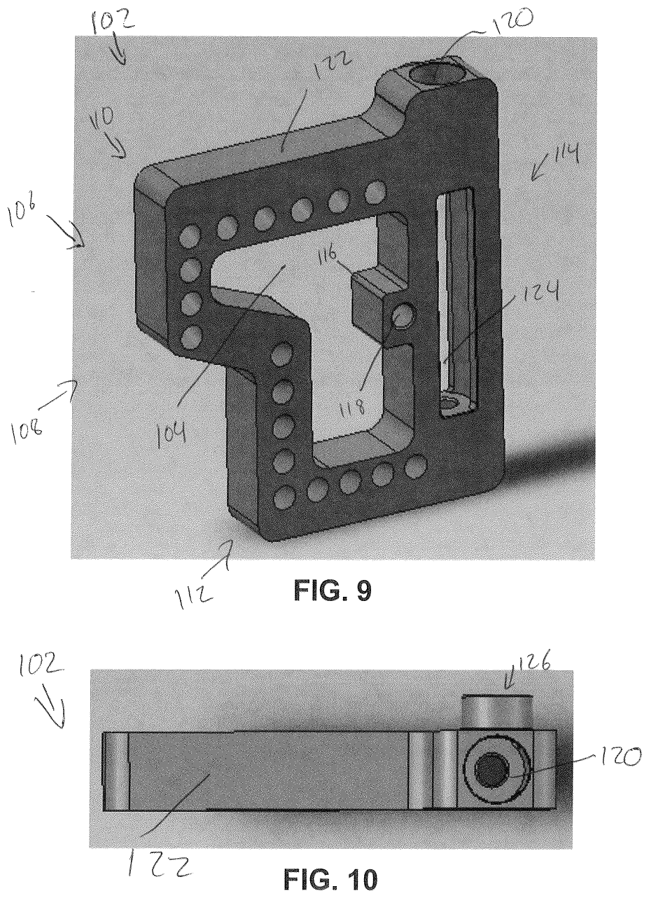

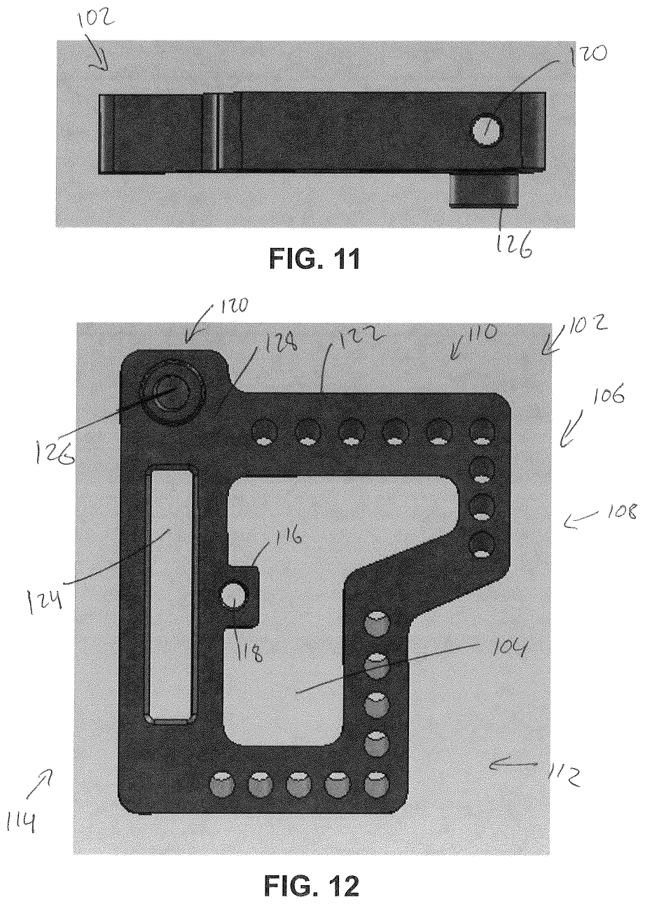

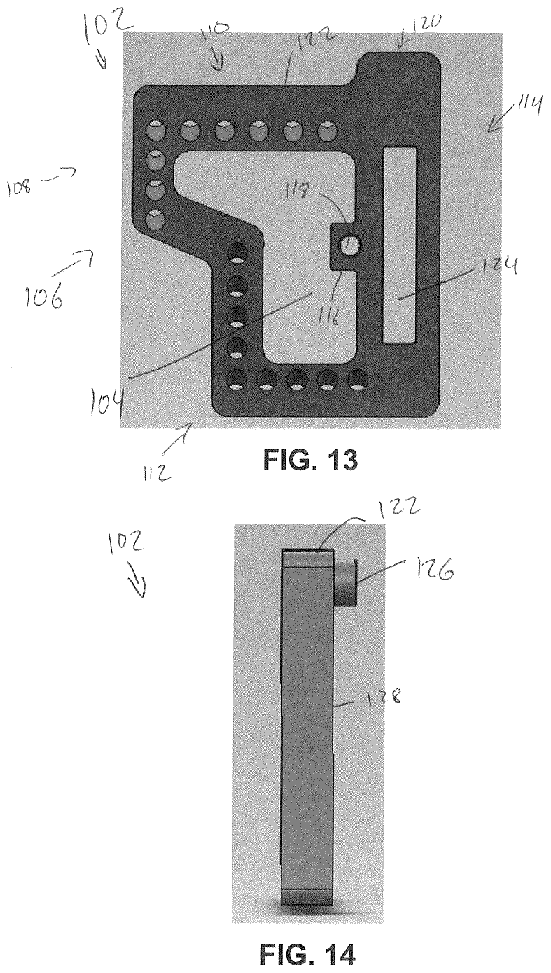

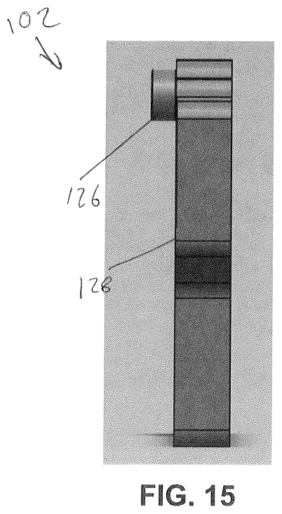

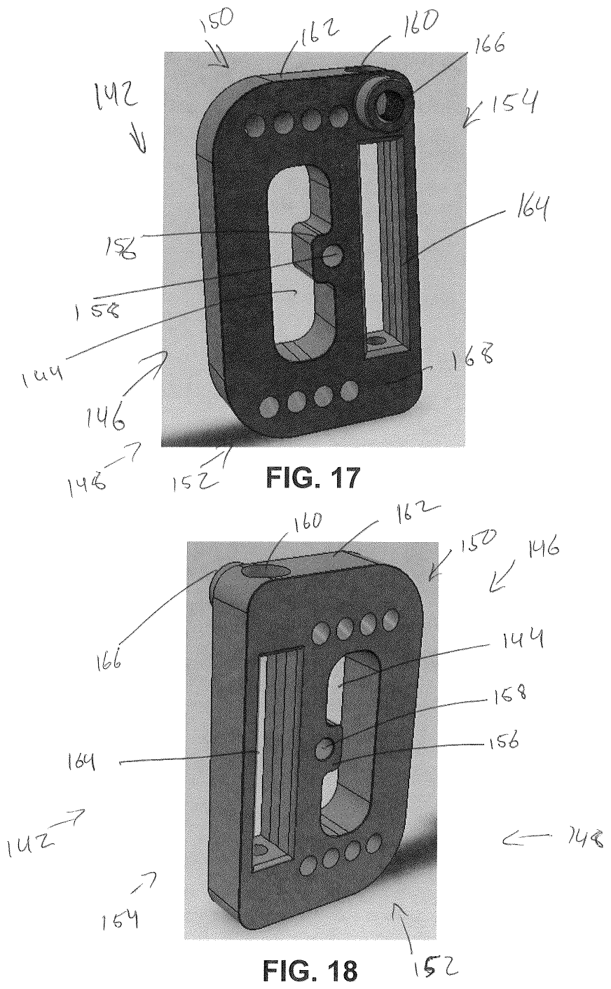

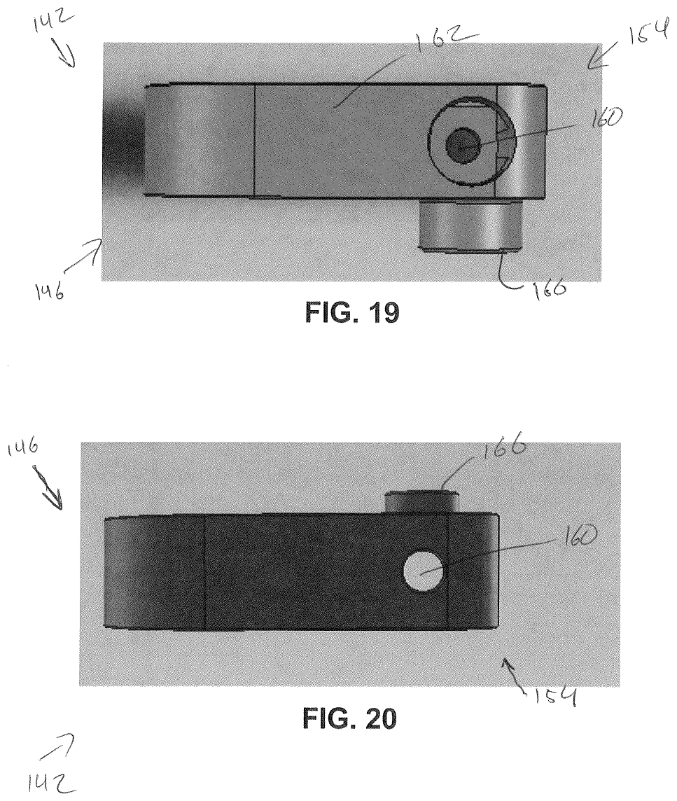

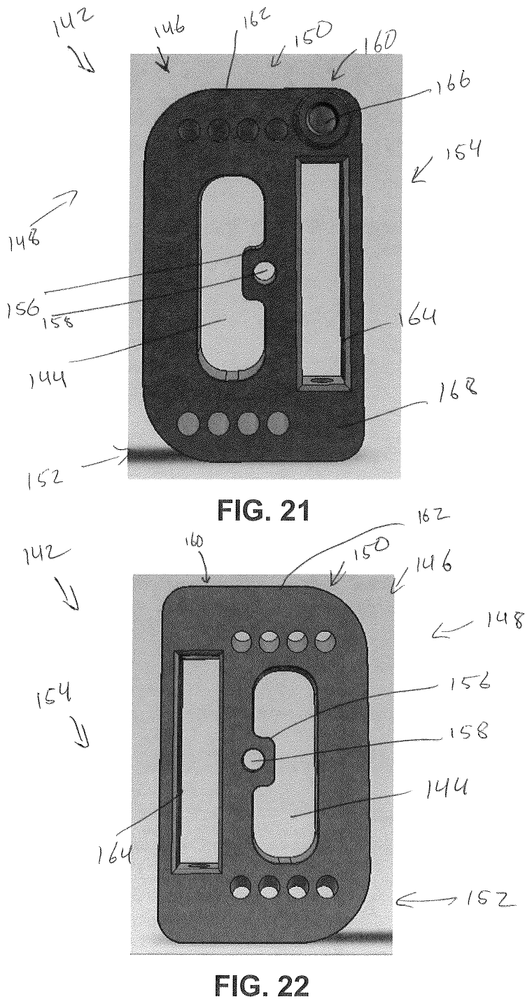

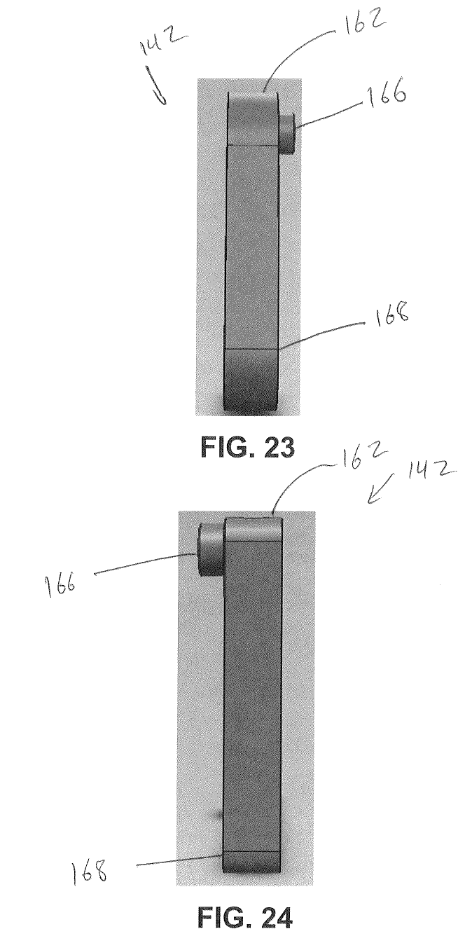

Referring now to FIGS. 1-26, an orthopedic implant system 100 and components thereof are shown, according to an exemplary embodiment. The system 100 and the components thereof may be implemented in accordance with the steps shown and described herein as an exemplary surgical method. It should be understood that, in implementing the system 100, additional instruments and components common to surgical systems and procedures may also be implemented. In some aspects, the system 100 may include duplicates of one or more components where one or multiples of the component are implemented in conjunction with the system 100. Further, it should be understood that in implementing the system 100, one or more of the components of the system 100 may be omitted.

Referring now to FIGS. 1-5, the system 100 is shown adjacent to a foot of a patient, according to an exemplary embodiment. As shown, the system 100 is positioned for implementation on a right foot for example, although the system 100 may also be implemented in an alternate configuration to that shown for use on a left foot of a patient. Further, the system 100 is shown for example to be positioned on a medial portion of the foot to accommodate a medial surgical approach, but may also be implemented on other portions of the foot (left or right) to accommodate alternate surgical approaches (e.g., implemented on the lateral side of the foot to accommodate a lateral surgical approach).

As shown in FIGS. 1-3, components of the system 100 including a first guide 102 (e.g., a “proximal” guide) and a second guide 142 (e.g., a “distal guide”) are positioned adjacent various bony anatomy on the medial side of the foot prior to coupling the first and second guides 102, 142 with the bony anatomy. For example, the first guide 102 may be positioned such that at least a portion thereof is adjacent the navicular 202 and the medial cuneiform 204 of the patient. In some aspects, the first guide may also be positioned adjacent a first metatarsal 206 and/or a calcaneus or talus of a patient, depending on the patient anatomy. The second guide 142 is shown to be positioned adjacent to the first metatarsal 206 of the patient, but in some aspects may be placed adjacent to additional or alternative anatomic structures, or at various points along the length of the first metatarsal.

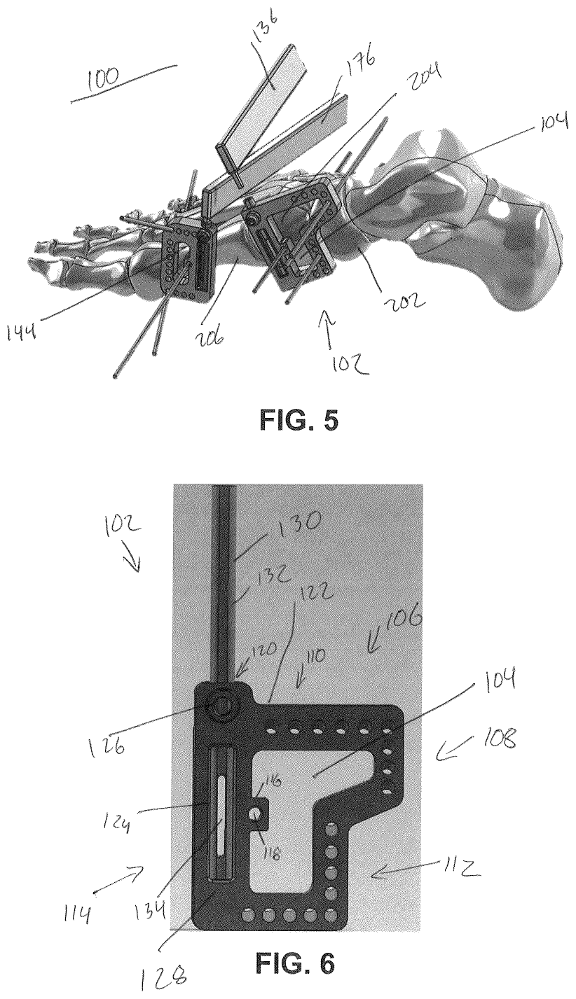

As shown in FIGS. 4-5, the first and second guides 102, 142 may be releasably coupled with the aforementioned anatomy adjacent to which they are positioned using one or more stabilization wires or pins (e.g., “k-wires”) inserted through various holes/openings in the first and second guides 102, 142. As shown, the first and second guides 102, 142 may be positioned and subsequently attached to the foot anatomy as shown at various angles or orientations according to a physician's preference. The system 100, as shown in FIGS. 4-5, may also include a pair of guide elements 136, 176 configured to releasably couple with the first and second guides 102, 142, respectively. In some aspects, the guide elements 136, 176 may be the same or similar to one another, and may include features such as those shown with respect to the guide element 176 as shown in FIG. 26.

The guide element 176 is seen to have an elongated body (rectangular prism-shaped as shown for example), and is shown to include a first opening 178 extending into the guide element 176 and positioned at a first end thereof. The guide element 176 may receive at least a portion of a component of the system 100 at least partially within the opening 178 so as to facilitate releasable coupling with the first and/or second guide 102, 142. The guide element 176 is further shown to include a pair of openings 180, positioned at an end of the guide element 176 from the opening 180, each configured to receive at least a portion of a radiopaque element at least partially therein. For example, the physician may insert a k-wire at least partially within one or both of the openings 180 such that the k-wire may represent a plane that when viewed under fluoroscopic imaging will facilitate positioning of the first and second guides 102, 142. In some aspects, the openings 180 may extend through the entirety of the guide element 176 such that k-wires may be inserted and/or removed at the same end as the opening 178 or at the opposite end.

Referring now to FIGS. 1-15, the first guide 102 is shown, according to an exemplary embodiment. The first guide 102 is shown to include an opening 104 disposed within a central portion thereof and defined by the geometry of the first guide 102. Further, the opening 104 is defined by a coupling portion 106 and a cutting portion 114 of the first guide 102 which, as shown in FIGS. 1-15, are integral with one another (but in some aspects may be modular and releasably couplable with one another). Collectively, the coupling portion 106 and the cutting portion 114 form a perimeter defining the opening 104 therein which, as shown, has a modified “7-shaped” or “L-shaped” geometry.

The coupling portion 106 is shown to include an array of holes 108, which includes a set of upper holes 110 and a set of lower holes 112 which extend from a front surface 128 of the coupling portion 106 through to the rear side thereby establishing fluid communication therebetween. As shown, the upper holes 110 and the lower holes 112 are incrementally spaced from one another (within their respective sets), and are separated from one another by a portion of the coupling portion 106 in which there are no holes. However, in some aspects the hole array 108 may be consistent from the upper holes 110 to the lower holes 112, without any space therebetween. The upper holes 110 are angled relative to the front surface 128 of the first guide 102 (e.g., such that the longitudinal axis of each of the holes is positioned at an oblique angle downward relative to the front surface 128 of the first guide 102, but in some aspects may be configured otherwise). In some aspects, the upper holes 110 may be angled upward or positioned such that the longitudinal axes of the holes are positioned orthogonally relative to the front surface 128. The lower holes 112 are angled relative to the front surface 128 of the first guide 102 (e.g., such that the longitudinal axis of each of the holes is positioned at an oblique angle upward relative to the front surface 128 of the first guide 102, but in some aspects may be configured otherwise). In some aspects, the lower holes 112 may be angled downward or positioned such that the longitudinal axes of the holes are positioned orthogonally relative to the front surface 128. The holes of the array of holes 108 may have alternate geometries configured to facilitate compatibility with other components of the system 100. As shown, the upper holes 110 extend further in a lateral direction (relative to the first guide 102, not anatomically) than the lower holes 112, but in some aspects the upper and lower holes 110, 112 may be positioned within similar footprints depending on the geometry of the coupling portion 106.

The cutting portion 114 is shown to have a substantially linear geometry when viewed from a front perspective view, and includes a protrusion 116 extending laterally from a side of the cutting portion 114 into the opening 104. As shown, the protrusion 116 has a substantially rectangular geometry, but in some aspects may have alternate geometries. The protrusion 116 includes an opening 118 extending therethrough, which may be similar in size and/or geometry to the holes of the hole array 108. In some aspects, the physician may place a k-wire through the opening 118 and into a desired bone to fix a central point of the first guide 102, and then subsequently place additional k-wires through one or more holes of the hole array 108 once the first guide 102 has been positioned at a desired angle (e.g., pivoted about the fixation wire through the opening 118).

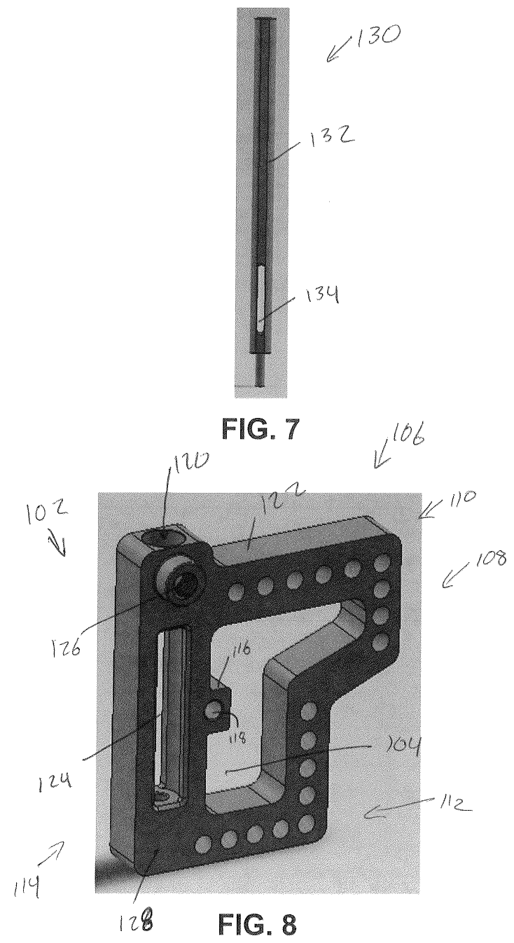

The cutting portion 114 is further shown to include a cannulation 120 extending from a top surface 122 (which may have elevation changes across the width of the first guide 102) through the cutting portion 114 to a bottom surface of the first guide 102 thereby establishing fluid communication therebetween. As shown in at least FIG. 10, the cannulation 120 may have multiple diameters along a length thereof including, for example, a greater diameter adjacent the top surface 122 than adjacent the bottom surface. With reference to FIGS. 1-6, the cannulation 120 is configured to receive at least a portion of an insert 130, which has a substantially elongated geometry as shown in FIG. 7. The insert 130 is shown to have a shaft 132, with a slot 134 disposed in and extending along a partial length of the shaft 132. Further, the shaft 132 is shown to include a first diameter along a first length thereof, and a second diameter along a second length thereof. The first length of the shaft 132 may be a majority of the length of the shaft 132, with the second length thereof a minority portion of the length of the shaft 132. The shaft 132 may have a substantially circular cross-sectional geometry along the first length thereof, which may for example, include one or more flats disposed along the geometry (e.g., opposite one another, equidistant from one another, etc.). Along the second length, the shaft 132 may also include a substantially circular cross-sectional geometry.

The insert 130 may be inserted at least partially within the cannulation 120, as shown in at least FIG. 6, such that the second length of the shaft 132 is received in the lower portion of the cannulation 120 having a lesser diameter than portions of the cannulation 120 there above. Similarly, when the insert 130 has been inserted in the cannulation 120 to an appropriate position, the slot 134 thereof will be positioned within a window 124 of the cutting portion 114 establishing fluid communication through the first guide 102 from the front surface 128 through to the bottom surface. Accordingly, a cutting instrument (e.g., saw, burr, etc.) may be inserted through the slot 134 and the window 124 simultaneously so as to access and cut bony anatomy positioned opposite the first guide 102 from the top surface 122. When the insert 130 is in the inserted position as shown in FIG. 6, the insert 130 may be rotated about its longitudinal axis (which, in the inserted position, may be substantially coaxial with that of the cannulation 120) so as to angle the slot 134 within the window 124 and guide cutting at angles/in planes forming oblique angles relative to the front surface 128.

The cutting portion 114 is further shown to include an opening 126 positioned on an upper portion thereof and extending through from the front surface 128 to the cannulation 120, thereby establishing fluid communication therebetween. As shown in at least FIG. 8, the opening 126 extends through a protrusion extending outward from the front surface 128, which may include a threading disposed on inner walls thereof to facilitate threadably engaging a screw or other threaded component. When the insert 130 is positioned within the cannulation as shown in FIG. 6, a screw or other component may be inserted within (and threadably engaged with the threads thereof) the opening 126 and advanced such that a first end of the screw or other component contacts and applies a force to the outer surface of the shaft 132, thereby retaining the insert 130 in a desired position (e.g., rotationally such that the slot 134 is angled as desired relative to the window 124 and the first guide 102).

The window 124 is shown to have a greater vertical dimension than a horizontal dimension, and extend through the cutting portion 114 from the front surface 128 through to the rear surface. As shown, the window 124 has a greater cross-sectional area, when viewed from a front perspective as in FIG. 6, than the slot 134 (although in some aspects, the cross-sectional areas may be equal or the slot 134 may have a greater cross-sectional area than the window 124).

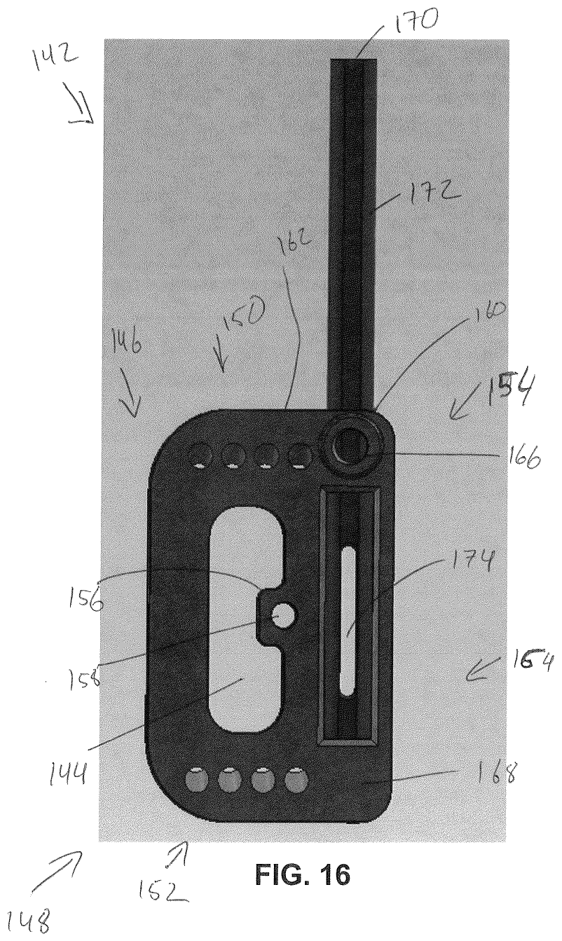

Referring now to FIGS. 16-25, the second guide 142 is shown, according to an exemplary embodiment. The second guide 142 is shown to include an opening 144 disposed within a central portion thereof and defined by the geometry of the second guide 142. Further, the opening 144 is defined by a coupling portion 146 and a cutting portion 154 of the second guide 142 which, as shown in FIGS. 16-25, are integral with one another (but in some aspects may be modular and releasably couplable with one another). Collectively, the coupling portion 146 and the cutting portion 154 form a perimeter defining the opening 144 therein which, as shown, for example as a modified “D-shaped” geometry.

The coupling portion 146 is shown to include an array of holes 148, which includes a set of upper holes 150 and a set of lower holes 152 which extend from a front surface 168 of the coupling portion 146 through to the rear side thereby establishing fluid communication therebetween. As seen, the upper holes 150 and the lower holes 152 are incrementally spaced from one another (within their respective sets), and are separated from one another by a portion of the coupling portion 146 through which there are no holes. However, in some aspects the hole array 148 may be consistent from the upper holes 150 to the lower holes 152, without any space therebetween. The upper holes 150 are angled relative to the front surface 168 of the second guide 142 (e.g., such that the longitudinal axis of each of the holes is positioned at an oblique angle downward relative to the front surface 168 of the second guide 142, but in some aspects may be configured otherwise). In some aspects, the upper holes 150 may be angled upward or positioned such that the longitudinal axes of the holes are positioned orthogonally relative to the front surface 168. The lower holes 152 are angled relative to a front surface 168 of the second guide 142 (e.g., such that the longitudinal axis of each of the holes is positioned at an oblique angle upward relative to the front surface 168 of the second guide 142, but in some aspects may be configured otherwise). In some aspects, the lower holes 152 may be angled downward or positioned such that the longitudinal axes of the holes are positioned orthogonally relative to the front surface 168. The holes of the array of holes 148 may have alternate geometries configured to facilitate compatibility with other components of the system 100. As shown, the upper holes 150 extend further in a lateral direction (relative to the second guide 142, not anatomically) than the lower holes 152, but in some aspects the upper and lower holes 150, 152 may be positioned within similar footprints depending on the geometry of the coupling portion 146.

The cutting portion 154 is shown to have a substantially linear geometry when viewed from a front perspective, and includes a protrusion 156 extending laterally from a side of the cutting portion 154 into the opening 144. As shown, the protrusion 156 has a substantially rectangular geometry, but in some aspects may have alternate geometries. The protrusion 156 includes an opening 158 extending therethrough, which may be similar in size and/or geometry to the holes of the hole array 148. In some aspects, the physician may place a k-wire through the opening 158 and into a desired bone to fix a central point of the second guide 142, and then subsequently place additional k-wires through one or more holes of the hole array 148 once the second guide 142 has been positioned at a desired angle (e.g., pivoted about the fixation wire through the opening 158).

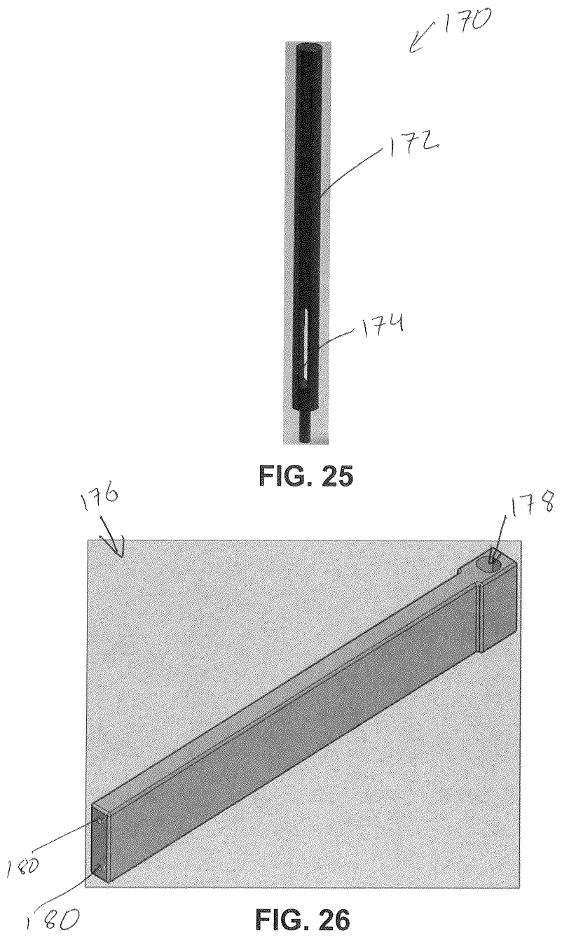

The cutting portion 154 is further shown to include a cannulation 160 extending from a top surface 162 (which may have elevation changes across the width of the second guide 142) through the cutting portion 154 to a bottom surface of the second guide 142 thereby establishing fluid communication therebetween. As shown in at least FIG. 10, the cannulation 160 may have multiple diameters along a length thereof including, for example, a greater diameter adjacent the top surface 162 than adjacent the bottom surface. With reference to FIGS. 1-6, the cannulation 160 is configured to receive at least a portion of an insert 170, which has a substantially elongated geometry as shown in FIG. 25. The insert 170 is shown to have a shaft 172, with a slot 174 disposed in and extending along a partial length of the shaft 172. Further, the shaft 172 is shown to include a first diameter along a first length thereof, and a second diameter along a second length thereof. The first length of the shaft 172 may be a majority of the length of the shaft 172, with the second length thereof a minority portion of the length of the shaft 172. The shaft 172 may have a substantially circular cross-sectional geometry along the first length thereof, which may include one or more flats disposed along the geometry (e.g., opposite one another, equidistant from one another, etc.). Along the second length, the shaft 172 may also include a substantially circular cross-sectional geometry.

The insert 170 may be inserted at least partially within the cannulation 160, as shown in at least FIG. 16, such that the second length of the shaft 172 is received in the lower portion of the cannulation 160 having a lesser diameter than portions of the cannulation 160 there above. Similarly, when the insert 170 has been inserted in the cannulation 160 to an appropriate position, the slot 174 thereof will be positioned within a window 164 of the cutting portion 154 establishing fluid communication through the second guide 142 from the front surface 168 through to the bottom surface. Accordingly, a cutting instrument (e.g., saw, burr, etc.) may be inserted through the slot 174 and the window 164 simultaneously so as to access and cut bony anatomy positioned opposite the second guide 142 from the top surface 162. When the insert 170 is in the inserted position as shown in FIG. 16, the insert 170 may be rotated about its longitudinal axis (which, in the inserted position, may be substantially coaxial with that of the cannulation 160) so as to angle the slot within the window 164 and guide cutting at angles/in planes forming oblique angles relative to the front surface 168.

The cutting portion 154 is further shown to include an opening 166 positioned on an upper portion thereof and extending through from the front surface 168 to the cannulation 160, thereby establishing fluid communication therebetween. As shown in at least FIG. 18, the opening 166 extends through a protrusion extending outward from the front surface 168, which may include a threading disposed on inner walls thereof to facilitate threadably engaging a screw or other threaded component. When the insert 170 is positioned within the cannulation as shown in FIG. 16, a screw or other component may be inserted within (and threadably engaged with the threads thereof) the opening 166 and advanced such that a first end of the screw or other component contacts and applies a force to the outer surface of the shaft 172, thereby retaining the insert 170 in a desired position (e.g., rotationally such that the slot 174 is angled as desired relative to the window 164 and the second guide 142).

The window 164 is shown to have a greater vertical dimension than a horizontal dimension, and extend through the cutting portion 154 from the front surface 168 through to the rear surface. As shown in FIG. 16, the window 164 has a greater cross-sectional area, when viewed from a front perspective, than the slot 174 (although in some aspects, the cross-sectional areas may be equal or the slot 174 may have a greater cross-sectional area than the window 164).

The terminology used herein for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An orthopedic instrument system, comprising:
a first cut guide;
a second cut guide; and
at least one insert that is at least partially receivable within at least a portion of the first cut guide and the second cut guide.

2. The orthopedic instrument system of claim 1, wherein the at least one insert comprises a first insert and a second insert.

3. The orthopedic instrument system of claim 2, wherein the first insert is at least partially receivable within at least a portion of the first cut guide, and wherein the second insert is receivable within at least a portion of the second cut guide.

4. The orthopedic instrument system of claim 3, wherein the first cut guide comprises:
a front surface opposite a rear surface; and
an opening extending through the first cut guide from the front surface to the rear surface.

5. The orthopedic instrument system of claim 4, wherein the first cut guide further comprises:
a plurality of cylindrical openings extending through the first cut guide from the front surface through to the rear surface.

6. The orthopedic instrument system of claim 5, wherein at least some of the plurality of cylindrical openings are positioned between the opening and one or more edges of the first cut guide.

7. The orthopedic instrument system of claim 6, wherein at least some of the plurality of cylindrical openings comprise central axes positioned at an oblique angle relative to the front and rear surfaces of the first cut guide.

8. The orthopedic instrument system of claim 6, wherein the first cut guide comprises a protrusion extending into the opening, wherein the protrusion comprises a cylindrical opening extending from a front surface of the protrusion through to a rear surface of the protrusion.

9. The orthopedic instrument system of claim 5, wherein the first cut guide further comprises:
a cannulation extending from a top surface of the first cut guide through to a bottom surface of the cut guide, wherein at least a portion of the at least one insert is receivable within the cannulation.

10. The orthopedic instrument system of claim 9, wherein the cannulation comprises:
a first diameter from the top surface of the first cut guide along a partial height of the cannulation to a point between the top surface of the first cut guide and the bottom surface of the cut guide; and
a second diameter from the point between the top surface of the first cut guide and the bottom surface of the first cut guide along the remaining height of the cannulation through to the bottom surface of the first cut guide.

11. The orthopedic instrument system of claim 10, wherein the first diameter is greater than the second diameter.

12. The orthopedic instrument system of claim 11, wherein the at least one insert comprises a substantially cylindrical geometry.

13. The orthopedic instrument system of claim 12, wherein the at least one insert further comprises an upper portion having a first diameter and a lower portion having a second diameter.

14. The orthopedic instrument system of claim 13, wherein the first diameter of the upper portion of the at least one insert is greater than the second diameter of the lower portion of the at least one insert.

15. The orthopedic instrument system of claim 14, wherein the first diameter of the upper portion on the at least one insert is lesser than the first diameter of the cannulation but greater than the second diameter of the cannulation.

16. The orthopedic instrument system of claim 15, wherein the second diameter of the lower portion of the at least one insert is lesser than the second diameter of the cannulation.

17. An orthopedic instrument system, comprising:
a first cutting guide, comprising:
a coupling portion comprising a plurality of openings extending from a front surface therethrough to a rear surface;
a cutting portion integral with the coupling portion, comprising:
a first cannulation extending from a top surface therethrough to a bottom surface, wherein the cannulation comprises an upper portion having a first diameter and a lower portion having a second diameter; and
a window extending from the front surface through to the rear surface and overlapping with the cannulation; and
a second cutting guide comprising a second cannulation; and
at least one insert receivable at least partially within the cannulation of the first and second guides, the insert comprising a slot extending therethrough.

18. The orthopedic instrument system of claim 17, wherein the first diameter of the upper portion of the first cannulation is greater than the second diameter of the lower portion of the first cannulation.

19. The orthopedic instrument system of claim 18, wherein the at least one insert comprises an upper portion having a first diameter and a lower portion having a second diameter, wherein the first diameter of the upper portion is greater than the second diameter o the lower portion.

20. An orthopedic instrument system, comprising:
a first cutting guide, comprising:
a coupling portion comprising a plurality of openings extending from a front surface therethrough to a rear surface;
a cutting portion integral with the coupling portion, comprising:
a cannulation extending from a top surface therethrough to a bottom surface; and
a window extending from the front surface through to the rear surface and overlapping with the cannulation; and
a second cutting guide, comprising:
a coupling portion comprising a plurality of openings extending from a front surface therethrough to a rear surface;
a cutting portion integral with the coupling portion, comprising:
a cannulation extending from a top surface therethrough to a bottom surface; and
a window extending from the front surface through to the rear surface and overlapping with the cannulation; and
at least one insert receivable at least partially within the cannulation of the first and second guides, the insert comprising a slot extending therethrough.
